(19) United States
(12) Patent Application Publication
(10) Pub. No.: US 2021/0282733 A1
(43) Pub. Date: Sep. 16, 2021

(54) EDGE NOISE REDUCTION

(71) Applicant: KONINKLIJKE PHILIPS N.V., EINDHOVEN (NL)

(72) Inventors: Bernhard Johannes BRENDEL, NORDERSTEDT (DE); Kevin Martin BROWN, CHARDON, OH (US)

(21) Appl. No.: 16/330,408

(22) PCT Filed: Sep. 1, 2017
(86) PCT No.: PCT/EP2017/072029

§ 371 Date: Mar. 5, 2019

Related U.S. Application Data
(60) Provisional application No. 62/394,480, filed on Sep. 14, 2016.

(30) Foreign Application Priority Data
Oct. 24, 2016 (EP) 16195197.5

(51) Int. Cl. A61B 6/00; G06T 5/00; G06T 7/00; G06T 5/50

(52) U.S. Cl. CPC A61B 6/5258; G06T 5/002; G06T 7/0012; G06T 2207/10116; A61B 6/469; G06T 2207/10081; G06T 5/50

(57) ABSTRACT

Present multi-spectral CT approaches are able to cancel the noise from the combined (mono-energy) image. However, medical professionals also find it useful to consult the basis images which are combined (summed) form the mono image, because they can provide useful extra diagnostic information. However, denoising of the basis images can lead to a “jagged” appearance of edges in the denoised basis images, inconveniently requiring further image processing steps to take place before the basis images can be clearly read. Accordingly, there is provided an apparatus (30) for simultaneous edge noise reduction. The apparatus comprises a processor (32). The processor is configured to receive first (s₀) and second (p₀) input image data, and to receive first (s) and second (p) denoised input image data. The first and second input image data contains noise which is anti-correlated between the first and the second input image data. The processor is further configured to generate uncorrelated noise data (m−m₀) using the first (s₀) and second (p₀) input image data and the first (s) and second (p) denoised input image data. The uncorrelated noise data (m−m₀) data represents uncorrelated noise between the first (s) and second (p) denoised input image data. The processor is further configured to generate output image data based on the uncorrelated noise data (m−m₀). The output image data has a reduced level of edge noise in comparison to the first and/or the second input image data.

### FIELD OF THE INVENTION

This invention relates generally to an apparatus configured to reduce edge noise in images, and more particularly to medical X-ray images obtained using a spectral X-ray approach. Also discussed are a medical image processing system, a simultaneous edge noise reduction method, a computer program element, and a computer readable medium.

### BACKGROUND OF THE INVENTION

There is an increased interest in applying image-based denoising algorithms to conventional, and multi-spectral CT images, to improve the image quality of filtered back-projection (FBP) imaging algorithms by reducing noise in the images. This can enable the reduction of the X-ray dose to a patient, or an improvement of image clarity at the same X-ray dose.

Many types of image-based denoising algorithms retain noise at the edges of objects in the denoised FBP images, especially when “edge preserving” penalty functions are used in the denoising algorithms. This causes objects displayed in medical images to appear to have “rough” edges, when in reality they would have smooth edges. This confuses medical professionals tasked with interpreting such images.

US 2016/0071245 A1 discusses a denoising approach for de-noised reconstructed image data edge improvement, but such approaches may be further improved.

### SUMMARY OF THE INVENTION

According to a first aspect, there is provided an apparatus for simultaneous edge noise reduction, comprising:

a processor.

The processor is configured to receive first and second basis image data, and to receive first and second denoised basis image data. The first and second basis image data has been obtained by decomposing multi-spectral image data of a region of interest of a patient onto first and second basis functions.

The first and second basis image data contains noise which is anti-correlated between the first and the second basis image data, respectively.

The processor is further configured to generate uncorrelated noise data using the first and second basis image data and the first and second denoised basis image data.

The uncorrelated noise data represents uncorrelated noise between the first and second denoised basis image data.

The processor is further configured to generate output image data based on the generated uncorrelated noise data, by applying a first weight to the first basis image data, and a second weight to the second basis image data, wherein the first and second weights function to remove uncorrelated noise data from the first and second basis image data.

The output image data is a mono-energy combination of the first basis image data weighted by the first weight, and the second basis image data weighted by the second weight, and has a reduced level of edge noise in comparison to the first and/or the second input image data.

Accordingly, it is proposed to process the first and second denoised input image data using only the uncorrelated noise. The anti-correlated noise of the images is not used to denoise the first and second denoised input image data. According to the first aspect, when reconstructing multi-spectral CT images according to the processing approach discussed herein, denoised first and second basis images (generated from a decomposition of the multi-spectral CT data onto a set of basis functions) are provided which do not have jagged edges. Therefore, the output image data contains image data which can be used by a medical professional directly, without further image processing to reduced jagged edges.

Owing to the fact that the output image data does not need to be further denoised, a reduced-complexity of image processing is possible.

In addition, denoised first and second output image data are scaled such that the sum of the denoised first and second output image data in the output image data at each stage of the processing represents always a “mono” image at a particular energy (the term “mono” is an abbreviation for the phrase “mono-energy”, e.g., at the effective X-ray energy of the CT system).

Thus, at all stages of processing, the reprocessed mono energy image, and the denoised first and second basis images are available for viewing by a medical professional at acceptable quality.

Optionally, the output image data is a mono-energy combination of the first basis image data weighted by the first weight, and the second basis image data weighted by the second weight.

According to an example, there is provided an apparatus for simultaneous edge noise reduction. The apparatus comprises:

a processor.

The processor is configured to receive first and second input image data, and to receive first and second denoised input image data. The first and second input image data contains noise which is anti-correlated between the first and the second input image data. The processor is further configured to generate uncorrelated noise data using the first and second input image data and the first and second denoised input image data.

The uncorrelated noise data represents uncorrelated noise between the first and second denoised input image data. The processor is further configured to generate output image data based on the uncorrelated noise data. The output image data has a reduced level of edge noise in comparison to the first and/or the second input image data.

According to a second aspect, there is provided a medical imaging system. The system comprises:

a medical image acquisition apparatus; and

a medical image processing apparatus comprising an apparatus for simultaneous edge noise reduction according to the first aspect or its optional embodiments.

The medical image acquisition apparatus is configured to acquire multi-spectral medical imaging data of a region of interest of a patient, and to provide the multi-spectral medical imaging data to an input of the medical image processing apparatus.

The medical image processing apparatus is configured to receive the multi-spectral medical imaging data, and to process it using the apparatus for simultaneous edge noise reduction.

The medical image processing apparatus is configured to generate output image data having a reduced level of edge noise.

Accordingly, a medical image processing system is provided which can display to a medical professional edge-improved first and second basis images alongside a combined image, with no further processing stages of the edge-improved first and second basis images needing to be made.

According to a third aspect, there is provided a simultaneous edge noise reduction method. The method comprises:

a) receiving first and second basis image data, wherein the first and second basis image data has been obtained by decomposing multi-spectral image data of a region of interest of a patient onto first and second basis functions;
b) receiving first and second denoised basis image data of the region of interest of a patient;

wherein the first and second basis image data contains noise which is anti-correlated between the first and the second basis image data, respectively;

c) generating uncorrelated noise data using the first and second basis image data and the first and second denoised basis image data;

wherein the uncorrelated noise data represents uncorrelated noise between the first and second denoised basis image data; and

d) generating output image data based on the generated uncorrelated noise data, by applying a first weight to the first basis image data, and a second weight to the second basis image data, wherein the first and second weights function to remove uncorrelated noise data from the first and second basis image data,

wherein the output image data is a mono-energy combination of the first basis image data weighted by the first weight, and the second basis image data weighted by the second weight, and has a reduced level of edge noise in comparison to the first and/or the second basis image data.

According to an example, there is provided a simultaneous edge noise reduction method. The method comprises:

a) receiving first and second input image data;
b) receiving first and second denoised input image data;
wherein the first and second input image data contains noise which is anti-correlated between the first and the second input image data;
c) generating uncorrelated noise data using the first and second input image data and the first and second denoised input image data,
wherein the uncorrelated noise data represents uncorrelated noise between the first and second denoised input image data; and
d) generating output image data based on the uncorrelated noise data,
wherein the output image data has a reduced level of edge noise in comparison to the first and/or the second input image data.

According to a fourth aspect, there is provided computer program element for controlling a processor and/or system as presented according to the first aspect or its optional embodiments, which, when the computer program element is executed by the processor and/or system, causes the processor and/or system to perform the method as discussed according to the third aspects or its optional embodiments.

According to a fifth aspect, there is provided a computer readable medium having stored the computer element of the fourth aspect.

In the following application, the term “simultaneous edge noise reduction” means that at least a pair of images having reduced edge-noise, such as a “photo” and a “scatter” image, are provided in the same processing step. No additional processing is necessary to generate edge reduction benefits in a combined (e.g., mono) image, or in the first and second basis images. A generally agreed measure of edge smoothness, for example, based on the Canny operator, applied both to images of the output image data and the denoised input image data, would find a lower amount of edge noise in the output image data.

In the following application, the term “uncorrelated noise data” refers to the difference between the sum of the first and second denoised input image data, and the sum of the first and second input image data.

In the following application, the terms “photo” and “scatter” data refer to the term of art used to identify decomposed basis image when the decomposition into photoelectric, and Compton scatter components is used. This is the approach discussed in Alvarez and Macovski in the paper “Energy-selective Reconstructions in X-ray Computerized Tomography”, in Phys. Med. Biol, 1976, Vol. 21, No. 5, 733-744. It will be appreciated that other basis functions can be used for the decomposition, such as “water” and “iodine”, for example. In the following application, the term “input image data” refers to the noisy multi-spectral image data (s₀ and p₀). In one example, such data may be original “photo” and “scatter” images generated by fitting multi-spectral energy data to a basis set of decomposition functions. Input image data s₀ and p₀ contain mutually anti-correlated noise generated by fitting multi-spectral energy data to a basis set of decomposition functions.

In the following application, the term “denoised input image data” refers to the multi-spectral image data denoted s and p which contains edge features having “jagged edges”. The “jagged edges” arise as a result of applying a denoising algorithm to image data containing mutually anti-correlated noise.

Therefore, it is a basic idea of the present application, when improving the edges in multi-spectral CT data, to avoid the usage of the anti-correlated part of the removed noise in denoised versions of the first and second basis images, and only to use the uncorrelated part of the noise in the denoised versions of the first and second basis images. Such a simultaneous edge noise reduction approach has the effect that, when applying the approach to first and second basis images (such as “photo” and “scatter” images), the subsequent summing of the first and second basis images to achieve a “mono” image (e.g., at the effective X-ray energy of the CT-system) is substantially identical to applying noise cancellation directly to the “mono” image from the beginning of the processing. In other words, the edge noise reduction can be simultaneously realized in the “photo”, “scatter”, and “mono” images, with no additional image processing being necessary. The same result holds for any other combination of “photo” and “scatter” image, for example the mono-energy image for other energies, material images, or the like.

### BRIEF DESCRIPTION OF THE DRAWINGS

Exemplary embodiments will be described with reference to the following drawings:

FIG. 1 illustrates a medical imaging system.

FIG. 2 illustrates a result of a prior art edge noise reduction method.

FIG. 3 illustrates an apparatus for simultaneous edge noise reduction according to the first aspect.

FIG. 4 illustrates an example of a result of a simultaneous edge reduction method according to the techniques discussed herein.

FIG. 5 illustrates a further example of a result of a simultaneous edge reduction method according to the techniques discussed herein.

FIG. 6 illustrates a method according to the second aspect.

### DETAILED DESCRIPTION OF EMBODIMENTS

In multi-spectral CT imaging, multi-spectral raw projection data is received from a CT imaging apparatus. The multi-spectral raw projection data contains information about the attenuation of a target at a plurality of energies. The multi-spectral raw projection data raw projection data is reconstructed into an image according to techniques known to the person skilled in the art. This may enable the provision, for example, of at least two input images representing the attenuation of two different X-ray energies at a region of interest of a target (patient), for example from a multi-spectral detection element.

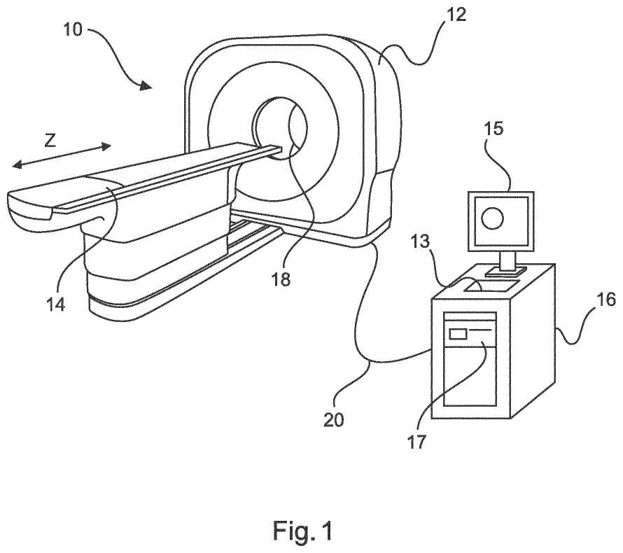

FIG. 1 illustrates a medical imaging system 10 such as a computed tomography (CT) scanner. The medical imaging system 10 includes an acquisition module 12, a patient support bed 14, and a processing computer 16. The acquisition module 12 comprises a generally stationary gantry and a rotatable gantry (not shown). The rotatable gantry rotates around an examination region 18 of the acquisition module 12. A region of interest of a patient may be positioned inside the examination region 18 along a z-axis of the patient support bed 14, to enable a medical image of the region of interest to be made.

The acquisition module 12 comprises a radiation source (not shown), such as an X-ray tube, rotatably supported by the rotatable gantry. The radiation source emits radiation which traverses the examination region 18. The beam may be formed into a cone, fan, wedge, or otherwise shaped radiation beam by collimators.

A one-dimensional (strip) or two-dimensional (planar) radiation sensor array (not shown) is positioned along an angular arc opposite the radiation source of the acquisition module 12 across the examination region 18. The radiation sensor array detects radiation crossing the examination region 18, and generates projection data indicative of the attenuation of the region of interest of the patient at a spatial position.

The acquisition module 12 is capable of generating multi-spectral CT data. In particular, the radiation sensor array may be a dual-layer multi-spectral radiation sensor array capable of simultaneous high and low energy discrimination. Alternatively, the acquisition module 12 is provided with two X-ray tubes, and two radiation detectors, with each of the two pairs of radiation tubes configured to emit X-ray radiation at different energies, and the detectors configured to receive X-ray radiation at different energies. Alternatively, the acquisition module 12 may be provided with one X-ray tube capable of fast kV energy switching. Alternatively, the acquisition module 12 is provided with a radiation detector capable of photon-counting. In essence, the acquisition module 12 is capable of providing multi-spectral CT data for processing.

Raw multi-spectral CT data obtained from a patient is communicated via a communication means 20 (such as a data cable, wireless link, fibre-optic cable, or Ethernet link) to a processing computer 16.

The processing computer 16 (processor) is configured to reconstruct the multi-spectral projection data collected by the acquisition module 12 to generate volumetric data of a region of interest of a patient. This can be achieved using a conventional filtered-back projection algorithm (FBP), a cone-beam algorithm, and iterative algorithm, or the like.

The processing computer 16 (processor) is configured to process the multi-spectral projection data using an energy-selective reconstruction approach, along the lines set out by Alvarez and Macovski in the paper “Energy-selective Reconstructions in X-ray Computerized Tomography”, in Phys. Med. Biol, 1976, Vol. 21, No. 5, 733-744. According to this technique, the attenuation of an X-ray may be represented as a function of energy by a small number of constants, by finding a set of basis functions such that the attenuation can be expressed as a linear combination of the basis functions. The choice of the basis functions is empirical, and in the Alvarrez paper an example is given in which the basis functions model the photoelectric interaction and the Compton scattering, thus generating “photo” and “scatter” images.

It will, however, be appreciated that other basis functions may be used in relation to the described technique representing the performance of water and iodine, or a wide range of materials useful to obtain a medical diagnosis from a multi-spectral CT image.

After reconstruction of the basis images (e.g. with filtered back-projection), denoising can be applied in the image domain. It can be applied to the decomposed photo-effect and scatter images, for example. In maximum-likelihood methods, both images can be denoised simultaneously, because the source of noise in both the decomposed photo-effect and scatter images is strongly anti-correlated, as a result of the originating basis function fitting process.

Attempts to apply the noise-cancellation method as described in US 2016/0071245 A1 to simultaneously denoised “photo” and “scatter” images from multi-energetic (spectral) systems can lead to unsatisfactory results.

It is a feature of the process of decomposing multi-spectral CT data onto basis functions that the noise between the decomposed basis images is anti-correlated. In other words, at a point in a first basis image having a noise component of a first magnitude, the same point in a second basis image will have a noise component of the same magnitude as that of the noise component in the first basis image (if both images are suitably scaled), but the noise component will have the opposite polarity compared to that of the first image. Attempts to denoise basis images using standard denoising algorithms leads to jagged edges in denoised versions of the decomposed basis images. Also, the sum (customarily known as a “mono image”) of the denoised decomposed basis images appears to have jagged edges. This causes confusion about whether or not a smooth object—for example a liver is affected by a disease or not. Thus, without further processing, the individually processed basis images provide little useful information to a medical professional and must be subjected to an extra step of image processing.

Previous noise cancellation techniques have exploited the fact that images with very smooth edges can be generated by adding back in, the negative of the noise which was removed by a denoising process, analogously to “noise-cancelling headphones”. Such methods are known as noise-cancellation methods.

The original noise-cancellation approach is based on equation (1):

m_{nc}=m−c(m₀−m)·∥∇m∥ (1)

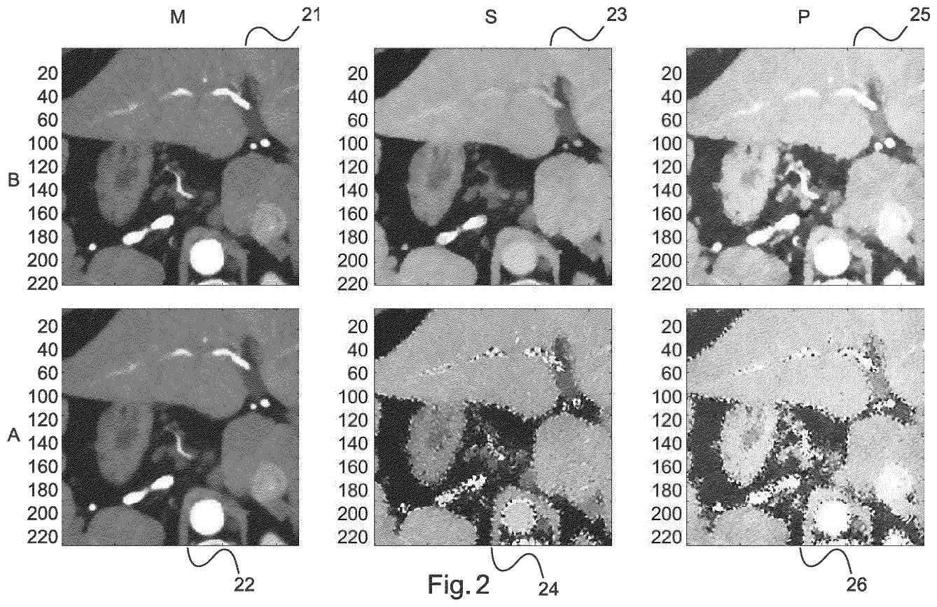

In equation (1), m is the denoised image, m₀ is the noisy input image, and ∇m is the gradient of the denoised image. The coefficient c is a parameter of the noise cancellation method that has to be adjusted to obtain a satisfactory reduction of jagged edges. The result m_{nc} is the output of the noise cancellation method (the “mono effect” image with reduced jagged edges). However, no factor c can be found that simultaneously reduces the jagged edges of first and second basis function images (for example, “photo” and “scatter”) of spectral CT data, as will be demonstrated in FIG. 2.

An insight applicable to the present invention is that first and second basis decomposition images (for example, “photo” and “scatter” images) should, at each stage of denoising processing, combine (sum) to form the mono-energy image at that stage of processing. It is assumed in the following that “photo” and “scatter” image are scaled such that they sum up to a “mono” image at the effective X-ray energy of the CT-system.

Developing this principle, the following relations can be developed:

m₀=s₀+p₀ (2a)

m=s+p (2b)

m_{nc}=s_{nc}+p_{nc} (2c)

In which (2a) denotes multi-spectral image data that has not been processed (denoised), (2b) denotes denoised multi-spectral image data, and in which (2c) denotes processed (final) image data. Substituting into equation (1):

m_{nc}=s−c(s₀−s)˜∥∇m∥+p−c(p₀−p)˜∥∇m∥ (3)

The two terms separated by the + operator on the right hand side of equation (3) are similar to the application of noise cancellation separately to first and second denoised basis images, for example, “photo” and “scatter images”. The difference, however, is that the norm of the gradient of m is used for local weighting of the differences (s₀−s) and (p₀−p).

FIG. 2 illustrates a result of such an approach. FIG. 2 shows multi-spectral CT mono (M), scatter (S), and photo (P) data before (B) and after (A) processing according to the approach of equation (3).

Thus, mono image 21 prior to processing shows a reduction of the jagged edges when mono image 22 after processing according to equation (3) is considered, as image 22 represents m_{nc}. However, it can be seen that denoised photo image 26 (p−c(p₀−p)·∥∇m∥) and denoised scatter image 24 (s−c(s₀−s)·∥∇m∥) both have jagged edges, and have not been successfully denoised, even though the same processing step did successfully reduce jagged edges in m_{nc}. In other words, a sum of two poor quality multi-spectral basis images can add up to provide a good quality mono image.

The reason for this is that a substantial amount of the noise energy in the “photo” and “scatter” images is anti-correlated, as a result of the underlying mathematics of the basis decomposition process. Thus, the noise (s₀−s and p₀−p) added back by the cancellation substantially cancels-out, when the basis images 24 and 26 are combined (summed) to generate the mono-energy image 22.

Especially for “photo” images, it has not been possible to reduce the jagged appearance of edges. Undesired jagged edges in medical images can confuse radiologists, because they begin to doubt the accuracy or reliability of other structures in an image, and because in some cases, rough organ boundaries can be an indication of disease. If an organ edge appears incorrectly rough or jagged, a “false positive” diagnosis of such a disease could result.

An insight of this application is that the anti-correlated part of the removed noise (s₀−s), and (p₀−p), should be avoided in the noise-cancellation of the first and second basis images (for example, “photo” and “scatter” images). Instead, only the uncorrelated part of the noise between the images should be used.

However, initially, only the combination (sum) of the uncorrelated parts of (s₀−s), and (p₀−p) is known: m₀−m. Thus, a method to split up m₀−m is now proposed, enabling the extraction of the uncorrelated parts of (s₀−s), and (p₀−p).

In this approach, (s₀−s) is substituted by:

(m₀−m)·∥∇s∥(∥∇s∥+∥∇p∥) (4a)

And (p₀−p) is substituted by:

(m₀−m)·∥∇p∥(∥∇s∥+∥∇p∥) (4b)

This is possible because the following relation holds:

$$( {m}_{0} - m ) \frac{‖ ∇ s ‖}{‖ ∇ s ‖ + ‖ ∇ p ‖} + ( {m}_{0} - m ) \frac{‖ ∇ p ‖}{‖ ∇ s ‖ + ‖ ∇ p ‖} = ( {s}_{0} - s ) + ( {p}_{0} - p ) = ( {m}_{0} - m ) \qquad ( 5 )$$

Thus, m_{nc} can also be expressed as equation (6):

$${m}_{n c} = s - c ( {m}_{0} - m ) \cdot \frac{‖ ∇ s ‖}{‖ ∇ s ‖ + ‖ ∇ p ‖} \cdot ‖ Vm ‖ + p - c ( {m}_{0} - m ) \cdot \frac{‖ ∇ p ‖}{‖ ∇ s ‖ + ‖ ∇ p ‖} \cdot ‖ ∇ m ‖ \qquad ( 6 )$$

Thus, according to a first aspect, an apparatus 30 for simultaneous edge noise reduction. The apparatus comprises:

a processor 32.

The processor is configured to receive first s₀ and second p₀ input image data, and to receive first s and second p denoised input image data.

The first and second input image data contains noise which is anti-correlated between the first and the second input image data.

The processor is further configured to generate uncorrelated noise data m−m₀ using the first s₀ and second p₀ input image data and the first s and second p denoised input image data.

The uncorrelated noise data m−m₀ data represents uncorrelated noise between the first s and second p denoised input image data.

The processor is further configured to generate output image data based on the uncorrelated noise data m−m₀, and the output image data has a reduced level of edge noise in comparison to the first and/or the second input image data.

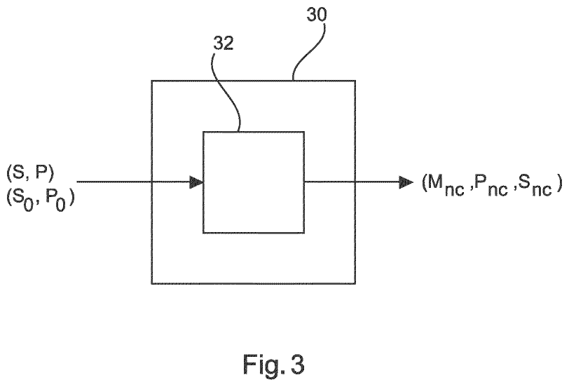

FIG. 3 illustrates an apparatus 30 according to the first aspect. The apparatus comprises a processor 32. The inputs to the apparatus are shown as the first s₀ and second p₀ input image data, and the received first s and second p denoised input image data. The output from the apparatus is shown as output image data which may comprise at least two of the edge-noise reduced mono m_{nc}, edge-noise-reduced first basis image and edge-noise-reduced second basis image.

The apparatus 30 is a means capable of performing image processing functions on digital image data. Optionally, the apparatus is implemented by a personal computer (PC), a dedicated graphics processor (GPU) of a personal computer, a hospital server system, or the like. The apparatus may use a form of hardware-acceleration such as an FPGA co-processor. Optionally, the apparatus may be hosted in a secure, encrypted “cloud” data processing center.

The first s₀ and second p₀ input image data to the apparatus 30 may be obtained from an acquisition module 12 during a medical image capturing process, as shown in FIG. 1. Alternatively, the input image data s₀ and second p₀ can be obtained from a hospital PACS system, a local area network, or a wide-area network, or from an encrypted and secure “cloud” server for the secure storage of medical images.

The first s and second p denoised input image data may optionally be generated in a precursor step by the apparatus 30 itself. In this embodiment, the first s and second p denoised input image data are generated by denoising algorithms as described, for example, in U.S. Ser. No. 13/508,751 entitled “Enhanced Image Data and Dose Reduction”.

Alternatively, the first s and second p denoised input image data may be received from a hospital PACS system or secure, encrypted “cloud” server for the secure storage of medical images.

The processor 32 is configured to generate the uncorrelated noise data m−m₀ generally according, for example, to the scheme of equations 2a and 2b, in which the first and second input image data is combined (summed), respectively, with the first and the second input image data.

The processor 32 is configured to generate output image data based on the uncorrelated noise data m−m₀ according to, for example, the scheme of equation 6. In other words, a first weight is applied to the first basis image data and a second weight is applied to the second basis image data. The first and second weights function to remove the uncorrelated noise data from the first and second basis image data.

The output image data has a reduced level of edge noise in comparison to the first and/or the second input image data. The processor 32 is optionally configured to provide the output image data in an imaging format, such as bitmap, or medically-specific lossless imaging standards.

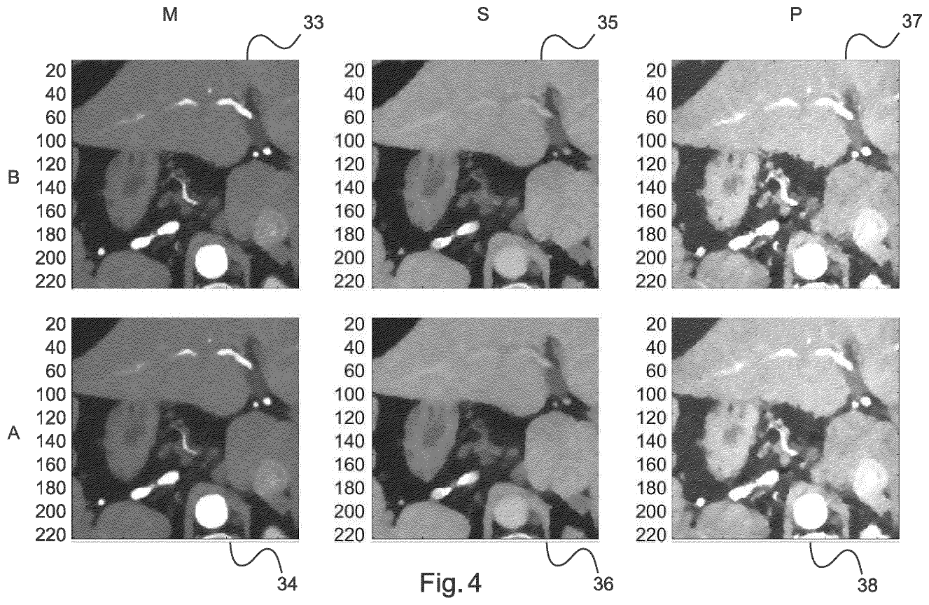

FIG. 4 illustrates an example of spectral CT data treated according to the technique of equation 6 above.

Image 34 shows m_{nc} after processing. Processed scatter image 36 represents a noisy scatter image 35 processed as output image data according to the relation

$$s - c ( {m}_{0} - m ) \cdot \frac{‖ ∇ s ‖}{‖ ∇ s ‖ + ‖ ∇ p ‖} \cdot ‖ ∇ m ‖ .$$

Processed photo image 38 represents a noisy photo image 37 processed as output image data according to the relation

$$p - c ( {m}_{0} - m ) \cdot \frac{‖ ∇ p ‖}{‖ ∇ s ‖ + ‖ ∇ p ‖} . ‖ ∇ m ‖ .$$

In comparison with the result of FIG. 4, it can be seen that the jagged edges of noisy scatter image 35 have been reduced in processed scatter image 36. It can also be seen that the jagged edges of noisy photo image 37 have been reduced in processed photo image 38. Unlike the result of FIG. 2, processed scatter image 36 and processed photo image 38 are, at a simultaneous step of processing, substantially free from jagged edge artefacts. Also notable is that the composite (sum) of the scatter and photo images, at the “before” (B) and “after” (A) stages of processing, is the same.

Thus, the proposed noise-cancellation approach significantly reduces the appearance of jagged edges in photo, scatter, and mono images, for example. However, the technique is generalizable to multi-spectral images decomposed onto any basis function.

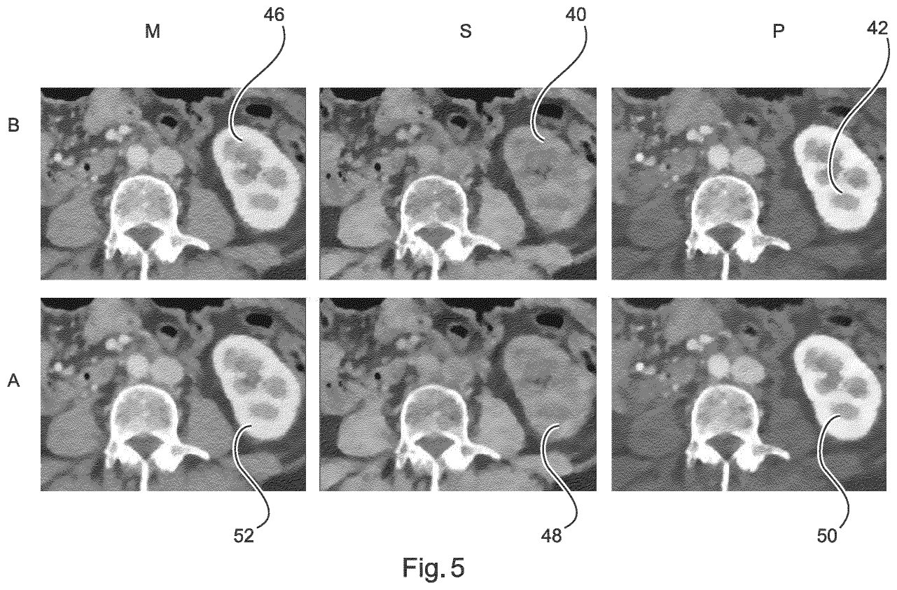

FIG. 5 illustrates a different image set to which the same technique as discussed above has been applied. The same labelling convention as for FIG. 4 is used. Thus the top-left image is a mono image before edge-reduction. The top-center image is a scatter image, before edge-reduction. The top-right image is a photo image, before edge-reduction. The bottom right image and bottom center images are photo images after simultaneous edge reduction by the algorithm discussed herein. The bottom left image is the mono energy image resulting from summing the bottom right image and bottom center images. It is clearly seen that an anatomical feature shown at organ 40 has a low level in the scatter image, but the same anatomical feature shows an increased photo response at area 42 in a photo image. Jagged edges are visible around this organ in the scatter and photo images. The scatter and photo images sum to reveal area 46 of the mono energy image before processing to reduce edge noise.

Following processing by the noise cancellation algorithm discussed above, it can be seen that the edges of areas 48 and 50 of the bottom-center scatter and bottom-right photo images, respectively, are less jagged than the areas 40 and 42 of the top-center and top-right images, respectively. The analogous area 52 of the bottom-right mono image constructed as the composite of the bottom-left photo image and the bottom-center scatter image shows that the sum of the scatter and photo image areas 48 and 50 gives a mono image having reduced edge noise, at the same step of processing.

Specific embodiments of the apparatus 30 will now be discussed.

Optionally, the output image data comprises a first output image having reduced edge noise in comparison to first denoised input image data.

Accordingly, a first output image (such a “photo” image) having a significantly reduced level of jagged edges is available to a medical professional without the need for further image processing.

Optionally, the output image data comprises a second output image having reduced edge noise in comparison to second denoised input image data.

Accordingly, a second output image (such a “scatter” image) having a significantly reduced level of jagged edges is available to a medical professional without the need for further image processing.

Optionally, the processor is further configured to combine the first output image and the second output image to form a composite output image.

Accordingly, formation of a “mono” image can be achieved from the first and second output images in a computational simple combination step (such as an addition, or a weighted addition).

Optionally, the processor is further configured to simultaneously display to a user two or more of the first output image, second output image, and the composite output image using an image viewing apparatus.

Accordingly, extra imaging information can be presented to a medical professional at a reduced level of image processing burden. Dependent on system design, this could enable reductions in image display latency when viewing a large sequence of multi-spectral CT images.

Optionally, the display modality may be a computer monitor, a tablet computer or smartphone, or a video screen, a virtual-reality headset, or a printed image.

Optionally, the composite output image m_{nc} is a weighted sum of the first output image s_{nc} and the second output image p_{nc} at each stage of processing.

Optionally, the processor is further configured to weight the uncorrelated noise data using a gradient-based weighting comprising the gradients of the first and second denoised input image data.

An example of a gradient-based weighting factor is

$$\frac{‖ ∇ s ‖}{‖ ∇ s ‖ + ‖ ∇ p ‖}$$

$$or$$

$$\frac{‖ ∇ p ‖}{‖ ∇ s ‖ + ‖ ∇ p ‖} .$$

In an embodiment, the uncorrelated noise of a first output image is provided by multiplying a mono energy image difference (m₀−m) with the factor

$$\frac{‖ ∇ s ‖}{‖ ∇ s ‖ + ‖ ∇ p ‖} .$$

In an embodiment, the uncorrelated noise of a second output image is provided by multiplying a mono energy image difference (m₀−m) with the factor

$$\frac{‖ ∇ p ‖}{‖ ∇ s ‖ + ‖ ∇ p ‖} .$$

Accordingly, noise between the first and second input image data (basis images) which is substantially uncorrelated is weighted using a gradient-based weighting algorithm.

Optionally, the gradient-based weighting comprises weighting the uncorrelated noise data using the gradient of the first denoised input image data, divided by a combination of the gradient of the first denoised input image data and the gradient of the second denoised input image data.

Optionally, the gradient-based weighting comprises weighting the uncorrelated noise data (m−m₀) using the gradient of the second denoised input image data (p), divided by a combination of the gradient of the first denoised input image data (s) and the gradient of the second denoised input image data (p).

Accordingly, the uncorrelated noise in respective first and second input image data (basis images) are is weighted using components derived from a combination of both the first and second input image data.

Optionally, the first and second basis functions are selected from the group of: (i) photo and scatter, (ii) water and iodine, or (iii) water and bone.

Optionally, the apparatus 30 is configured to denoise the received first s₀ and second p₀ input image data to provide first s and second p denoised input image data. In this case, the apparatus 30 does not need to be configured to receive firsts and second p denoised input image data.

In other words, the apparatus may be configured to pre-process the received first s₀ and second p₀ input image data.

Accordingly, the technique has broad application over many multi-spectral decomposition modalities.

According to a second aspect, medical imaging system 10 is provided. The system comprises:

a medical image acquisition apparatus 12; and

a medical image processing apparatus 16 comprising an apparatus 17 for simultaneous edge noise reduction according to the first aspect or its embodiments.

The medical image acquisition apparatus is configured to acquire multi-spectral medical imaging data of a region of interest of a patient, and to provide the multi-spectral medical imaging data to an input of the medical image processing apparatus.

The medical image processing apparatus is configured to receive the multi-spectral medical imaging data, and to process it using the apparatus for simultaneous edge noise reduction.

The medical image processing apparatus is configured to generate output image data having a reduced level of edge noise.

FIG. 1 illustrates a medical imaging system 10 according to the second aspect comprising a computed tomography (CT) scanner which has already been discussed above.

Optionally, the display apparatus 15 enables display of the output image data on a screen. Optionally, an interface means 13 (such as a keyboard, or a computer mouse), enables searching through a sequence of noise-cancelled multi-spectral data. In one embodiment, the noise-cancelled “mono-energy” image m_{nc} may be displayed in combination with the edge-noise improved first output image, as discussed above. In another embodiment, the noise-cancelled “mono-energy” image m_{nc} may be displayed in combination with the edge-noise improved second output image, as discussed above. In another embodiment, the noise-cancelled “mono-energy” image m_{nc} may be displayed in combination with both the edge-noise improved first and second output images.

Optionally, the apparatus 17 for simultaneous edge noise reduction is configured to store the processed output image data on a PACS system connected to the apparatus 17 via a local area network, for example.

According to a third aspect, a simultaneous edge noise reduction method is provided. The method comprises:

a) receiving first s₀ and second p₀ basis image data, wherein the first and second basis image data has been obtained by decomposing multi-spectral image data of a region of interest of a patient onto first and second basis functions;
b) receiving first s and second p denoised basis image data of the region of interest of a patient;

wherein the first and second basis image data contains noise which is anti-correlated between the first and the second basis image data, respectively;

c) generating uncorrelated noise data using the first s₀ and second p₀ basis image data and the first s and second p denoised basis image data;

wherein the uncorrelated noise data represents uncorrelated noise between the first s and second p denoised basis image data; and

d) generating output image data based on the generated uncorrelated noise data, by applying a first weight to the first basis image data, and a second weight to the second basis image data, wherein the first and second weights function to remove uncorrelated noise data from the first and second basis image data;

wherein the output image data is a combination of the first basis image data weighted by the first weight, and the second basis image data weighted by the second weight, and has a reduced level of edge noise in comparison to the first and/or the second basis image data.

Thus, at all stages of processing, the reprocessed “mono” image, and the denoised first and second basis images are available for viewing by a medical professional at acceptable quality.

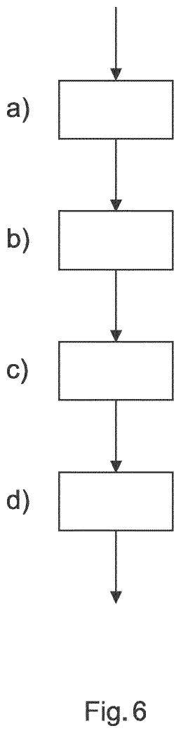

FIG. 6 illustrates the method according to the second aspect.

According to an embodiment of the third aspect, the output image data comprises a first output image s_{nc} having reduced edge noise in comparison to first denoised basis image data s.

According to an embodiment of the third aspect, the output image data comprises a second output image p_{nc} having reduced edge noise in comparison to second denoised basis image data p.

According to an embodiment of the third aspect, the method further comprises:

d1) combining the first output image s_{nc} and the second output image p_{nc} to form a composite output image m_{nc}.

According to an embodiment of the third aspect, the method further comprises:

d2) simultaneously displaying to a user two or more of the first output image s_{nc}, second output image p_{nc}, and the composite output image m_{nc} on an image viewing apparatus.

According to an embodiment of the third aspect, the composite output image m_{nc} is a weighted sum of the first output image s_{nc} and the second output image p_{nc} at each stage of processing.

According to an embodiment of the third aspect, the method further comprises:

d3) weighting the uncorrelated noise data m−m₀ using a gradient-based weighting of the firsts and second p denoised basis image data.

According to an embodiment of the third aspect, the gradient-based weighting comprises weighting the uncorrelated noise data m−m₀ using the gradient of the first denoised basis image data s, divided by a combination of the gradient of the first denoised basis image data s and the gradient of the second denoised basis image data p.

According to an embodiment of the third aspect, the gradient-based weighting comprises weighting the uncorrelated noise data m−m₀ using the gradient of the second denoised basis image data p, divided by a combination of the gradient of the first denoised basis image data s and the gradient of the second denoised basis image data p.

According to an embodiment of the third aspect, the first and second basis functions are selected from the group of: (i) photo and scatter, (ii) water and iodine, or (iii) water and bone.

According to an embodiment of the third aspect, there is the step of:

a1) denoising the received first s₀ and second p₀ basis image data to provide first s and second p denoised basis image data.

In this embodiment, step b) of receiving first s and second p denoised basis image data may, optionally, be omitted because the first s and second p denoised basis image data is instead generated (pre-processed) from the first s₀ and second p₀ basis image data.

According to this embodiment, the first and second basis image data and first so and second p₀ denoised basis image data acquired from a medical image acquisition apparatus 12 is pre-processed. It will, however, be appreciated that such pre-processed data will contain noise which is a mixture of correlated and anti-correlated noise, and that further image processing will be required to remove the noise.

It will be noted that the above-stated method steps may also be executed in a different order, particularly in a post-processing environment where instantaneous processing and display of images is not necessary.

The invention has been exemplified in relation to a multi-spectral CT imaging system, but it will be appreciated that the technique may be applied to other imaging approaches involving multi-spectral X-ray detectors, for example, breast cancer scanning machines, CT/PET approaches, and the like.

According to a fourth aspect, there is provided a computer program element for controlling a processor and/or system as claimed in the first aspect or its optional embodiments, which, when the computer program element is executed by the processor and/or system, causes the processor and/or system to perform the method of the second aspect or its optional embodiments.

According to a fifth aspect, there is provided a computer readable medium having stored the computer element of the fourth aspect.

In another aspect of the present invention, a computer program, or a computer program element, is provided that is characterized by being adapted to execute the method steps of the method of the second aspect, or its embodiments, as discussed according to one of the preceding embodiments, on an appropriate system.

The computer program element might therefore be stored on a computer unit, which might also be part of an embodiment of the present invention. This computing unit may be adapted to perform or induce a performance of the steps described above. Moreover, it may be adapted to operate the components of the above-described apparatus. The computing unit can be adapted to operate automatically, and/or to execute the orders of a user. A computer program may be loaded into the working memory of a data processor. The data processor may, thus, be equipped to carry out the method of the second aspect.

This exemplary embodiment of the invention covers both a computer program which is configured to use the invention initially, or a computer program that is configured from an existing program into a program that uses the invention by means of a software update, for example.

The computer program element is thus able to provide all necessary steps necessary to fulfil the procedure required according to the second aspect discussed above.

According to a further exemplary embodiment of the present invention, a computer readable medium, such as a CD-ROM, is presented; wherein the computer readable medium has a computer readable medium has a computer program element stored on it, wherein the computer program element is described in the previous section.

A computer program may be stored and/or distributed on a suitable medium, such as an optical storage medium or a solid-state medium supplied together with, or as part of other hardware. The computer readable medium may also be distributed in other forms, such as via the internet, or other wired or wireless telecommunication systems.

The computer program can also be presented over a network like the World Wide Web, and can be downloaded into the working memory of a data processor from such a network. According to a further exemplary embodiment of aspects of the present invention, a medium for making a computer program element available for downloading is provided, which computer program element is arranged to perform a method according to one of the previously described embodiments of the invention.

It should be noted that embodiments of the invention are described with reference to different subject-matter. In particular, some embodiments are described with reference to method-type features, whereas other embodiments are described with respect to apparatus-type features. A person skilled in the art will gather from the above, and following description, that, unless otherwise notified, in addition to any combination of features belonging to one type of subject-matter, also any combination of features belonging to one type of subject-matter, also any combination between features relating to different subject-matter is considered to be disclosed within this application.

All features can be combined to provide a synergetic effect, which is more than the simple summation of the features.

Whilst the invention has been illustrated and described in detail in the drawings and the foregoing description, such illustration and description are to be considered to be illustrative or exemplary, and not restrictive. The invention is not limited to the disclosed embodiments.

Other variations to the disclosed embodiments can be understood, and effected, by those skilled in the art in practicing the claimed invention, from a study of the disclosure in the drawings, the description, and the dependent claims.

In the claims, the word “comprising” does not exclude other elements or steps. The indefinite article “a” or “an” does not exclude a plurality. A single processor, or other unit, may fulfil the functions of several items recited in the claims. The mere fact that certain measures are recited in mutually different dependent claims does not indicate that a combination of these measures cannot be used to advantage.

Any reference signs in the claims should not be construed as limiting the scope of the claims.

1. An apparatus for simultaneous edge noise reduction, comprising:
a processor;
wherein the processor is configured to receive first and second basis image data, and to receive first and second denoised basis image data, wherein the first and second basis image data has been obtained by decomposing multi-spectral image data of a region of interest of a patient onto first and second basis functions;
wherein the first and second basis image data contains noise which is anti-correlated between the first and the second basis image data, respectively;
wherein the processor is further configured to generate uncorrelated noise data using the first and second basis image data and the first and second denoised basis image data,
wherein the uncorrelated noise data represents uncorrelated noise between the first and second denoised basis image data;
wherein the processor is further configured to generate output image data based on the generated uncorrelated noise data, by applying a first weight to the first basis image data, and a second weight to the second basis image data, wherein the first and second weights function to remove uncorrelated noise data from the first and second basis image data; and
wherein the output image data is a combination of the first basis image data weighted by the first weight, and the second basis image data weighted by the second weight, and has a reduced level of edge noise in comparison to the first and/or the second input image data.

2. The apparatus according to claim 1,
wherein the output image data comprises a first output image having reduced edge noise in comparison to first denoised basis image data.

3. The apparatus according to claim 1,
wherein the output image data comprises a second output image having reduced edge noise in comparison to second denoised basis image data.

4. The apparatus according to claim 1,
wherein the processor is further configured to combine the first output image and the second output image to form a composite output image.

5. The apparatus according to claim 4,
wherein the processor is further configured to simultaneously display to a user two or more of the first output image, second output image, and the composite output image using an image viewing apparatus.

6. The apparatus according to claim 4,
wherein the composite output image s a weighted sum of the first output image and the second output image at each stage of processing.

7. The apparatus according to claim 1,
wherein the processor is further configured to weight the uncorrelated noise data using a gradient-based weighting comprising the gradients of the first and second denoised basis image data.

8. The apparatus according to claim 7,
wherein the gradient-based weighting comprises weighting the uncorrelated noise data using the gradient of the first denoised basis image data, divided by a combination of the gradient of the first denoised basis image data and the gradient of the second denoised basis image data.

9. The apparatus according to claim 7,
wherein the gradient-based weighting comprises weighting the uncorrelated noise data using the gradient of the second denoised basis image data, divided by a combination of the gradient of the first denoised basis image data and the gradient of the second denoised basis image data.

10. The apparatus according to claim 1,
wherein the first and second basis functions are selected from the group of: (i) photo and scatter, (ii) water and iodine, or (iii) water and bone.

11. A medical imaging system comprising:
a medical image acquisition apparatus; and
a medical image processing apparatus comprising an apparatus for simultaneous edge noise reduction according to claim 1;
wherein the medical image acquisition apparatus is configured to acquire multi-spectral medical imaging data region of a interest of a patient, and to provide multi-spectral medical imaging data to an input of the medical image processing apparatus; and
wherein the medical image processing apparatus is configured to receive the multi-spectral medical imaging data, and to process it using the apparatus for simultaneous edge noise reduction; and
wherein the medical image processing apparatus is configured to generate output image data having a reduced level of edge noise.

12. A simultaneous edge noise reduction method, comprising:
a) receiving first and second basis image data, wherein the first and second basis image data has been obtained by decomposing multi-spectral image data of a region of interest of a patient onto first and second basis functions;
b) receiving first and second denoised basis image data of the region of interest of a patient;
wherein the first and second basis image data contains noise which is anti-correlated between the first and the second basis image data, respectively;
c) generating uncorrelated noise data using the first and second basis image data and the first and second denoised basis image data;
wherein the uncorrelated noise data represents uncorrelated noise between the first and second denoised basis image data; and
d) generating output image data based on the generated uncorrelated noise data, by applying a first weight to the first basis image data, and a second weight to the second basis image data, wherein the first and second weights function to remove uncorrelated noise data from the first and second basis image data,
wherein the output image data is a combination of the first basis image data weighted by the first weight, and the second basis image data weighted by the second weight, and has a reduced level of edge noise in comparison to the first and/or the second basis image data.

13. The simultaneous edge noise reduction method of claim 12, further comprising:
weighting the uncorrelated noise data using a gradient-based weighting comprising the gradients of the first and second denoised basis image data.

14. A computer program element for controlling a processor and/or system, which, when the computer program element is executed by the processor and/or system, causes the processor and/or system to perform the method of claim 12.

15. A computer readable medium having stored the computer element of claim 14.
